(19) United States
(12) Patent Application Publication
(10) Pub. No.: US 2024/0062911 A1
(43) Pub. Date: Feb. 22, 2024

(54) METHOD TO PREDICT HEART AGE

(71) Applicant: VUNO Inc., Seoul (KR)

(72) Inventors: Sunghoon JOO, Seoul (KR); Mineok CHANG, Seoul (KR); Kyung Geun KIM, Seoul (KR); Sungjae LEE, Seoul (KR); Yeongyeon NA, Seoul (KR)

(21) Appl. No.: 18/355,246

(22) Filed: Jul. 19, 2023

(30) Foreign Application Priority Data
Aug. 18, 2022 (KR) 10-2022-0103387

(51) Int. Cl. G16H 50/30; G06N 3/09; G06N 3/096; G06N 3/045

(52) U.S. Cl. CPC G16H 50/30; G06N 3/09; G06N 3/096; G06N 3/045

(57) ABSTRACT

An exemplary embodiment of the present disclosure discloses a method of estimating a heart age by using a pre-trained artificial neural network model. In particular, according to the present disclosure, a computing device obtains vital sign data of a user. The computing device estimates a heart age of the user based on the vital sign data of the user by using a pre-trained artificial neural network model. The pre-trained artificial neural network model corresponds to an artificial neural network model pre-trained based on information related to a heart disease.

### CROSS-REFERENCE TO RELATED APPLICATIONS

This application claims priority to and the benefit of Korean Patent Application No. 10-2022-0103387 filed in the Korean Intellectual Property Office on Aug. 18, 2022, the entire contents of which are incorporated herein by reference.

### BACKGROUND

### Technical Field

The present disclosure relates to a method of predicting a heart age, and particularly, to a method of predicting a heart age by inputting vital sign data of a user to an artificial neural network model pre-trained based on information related to a heart disease.

### Description of the Related Art

There is a growing body of research on how to detect and predict various cardiovascular diseases by using vital sign data, such as pulse waveforms (PPG) and electrocardiograms (ECG). However, most of these methods only provide classification results for specific types of cardiovascular disease and do not provide information about the overall heart health state of a user.

Korean Patent No. 2309022 (Sep. 29, 2021) discloses a remote monitoring system for vital signs based on artificial intelligence.

### BRIEF SUMMARY

The inventors have realized that heart age, on the other hand, may be used as an indicator that can provide information about the overall health state of the heart. Heart age may provide intuitive information about the overall health state of a user's heart through a difference from a user's chronological age. For example, when the estimated heart age is greater than the user's chronological age, this may be used as information that the heart is in poor health state compared to the user's chronological age and that the user is at a relatively high risk of cardiovascular disease.

As the means for measuring a heart age in the related art, measurement methods of indirectly calculating a heart age of the user by determining whether the user has risk factors for cardiovascular disease and then plugging the risk factors into a predetermined formula, or invasive measurement methods have been devised. However, the inventors have realized that these methods are not only inaccurate in estimating heart age, but also cannot provide multifaceted analytics, such as the possibility of heart disease in relation to a heart age of the user and predictions of future changes in heart age.

Accordingly, a need exists in the art for a method of predicting heart age that is more accurate and non-invasive than the prior art, and for a method of complexly combining heart age information with related information to provide information about the overall health state of a user's heart.

Various embodiments of the present disclosure is conceived in response to the foregoing background art, and aims to more accurately estimating a heart age of a user by receiving vital sign data of the user, analyzing information on the estimated heart age, and generating prediction information about a future heart age through an artificial neural network model pre-trained based on information related to heart disease.

An exemplary embodiment of the present disclosure for implementing the foregoing technical object discloses a method performed by a computing device for estimating a heart age. The method includes: obtaining vital sign data of a user; and estimating a heart age of the user based on the vital sign data by using a pre-trained artificial neural network model, and the pre-trained artificial neural network model may correspond to an artificial neural network model pre-trained based on information related to a heart disease.

In an alternative exemplary embodiment, the pre-trained artificial neural network model may correspond to an artificial neural network model pre-trained by supervised learning, and training data for the supervised learning may include: input data including measured vital sign data; and a ground-truth label including age information of the user associated with the measured vital sign data.

In the alternative exemplary embodiment, the pre-trained artificial neural network model may correspond to an artificial neural network model trained through transfer learning based on a heart disease model implemented to predict heart disease.

In the alternative exemplary embodiment, the pre-trained artificial neural network model may correspond to an artificial neural network model transfer-trained based on operations of: obtaining the train-completed heart disease model; and tuning a weight of the heart disease model by using training data associated with age estimation.

In the alternative exemplary embodiment, the pre-trained artificial neural network model may include a plurality of artificial neural network models trained through transfer learning based on a plurality of heart disease models implemented to predict a plurality of heart diseases, respectively, and an architecture of the pre-trained artificial neural network model may include an architecture of ensembling the plurality of artificial neural network models.

In the alternative exemplary embodiment, the architecture of ensembling the plurality of artificial neural network models may include an artificial neural network architecture for designating a plurality of probabilities for the plurality of heart diseases as weights, and weighted averaging output values of the plurality of artificial neural network models by using the weights.

In the alternative exemplary embodiment, the method may further include generating analytical information about the heart age of the user estimated by the pre-trained artificial neural network model, and the analytical information may include: information about the heart age of the user estimated by the pre-trained artificial neural network model; information about a position of the user in a distribution of heart ages of users in the same age group; and information about a comparison with a group of users with major heart diseases.

In the alternative exemplary embodiment, the method may further include generating predictive information about a future heart age of the user based on the estimated heart age of the user by using the pre-trained artificial neural network model.

In the alternative exemplary embodiment, the generating of the predictive information about the future heart age of the user based on the estimated heart age of the user by using the pre-trained artificial neural network model may include: checking whether past vital sign data of the user exists; and generating predictive information about a future heart age based on the estimated heart age information and past vital sign data by using the pre-trained artificial neural network model.

In the alternative exemplary embodiment, the past vital sign data may be measured at different time intervals.

In the alternative exemplary embodiment, the pre-trained artificial neural network model may correspond to an artificial neural network model pre-trained by supervised learning, and the training data for the supervised learning including: input data including a plurality of vital sign data measured over a specific time period; and a ground-truth label that includes heart age information associated with a time point after a predetermined time from the specific time period, to output predictive information about a future heart age.

correspond to an artificial neural network model trained with the training data for the supervised learning including: input data including a plurality of vital sign data measured over a specific time period; and a ground-truth label that includes heart age information associated with a time point after a predetermined time from the specific time period, to output predictive information about a future heart age.

In the alternative exemplary embodiment, the pre-trained artificial neural network model may include at least one of a gated recurrent unit and a long short-term memory.

In the alternative exemplary embodiment, the pre-trained artificial neural network model may correspond to an artificial neural network model pre-trained with at least one of operations of interpolating (imputating) the past vital sign data of the user by using an interpolation network and applying the interpolated past vital sign data to learning; or introducing a decay rate into an input layer and a hidden layer of the artificial neural network model.

Another exemplary embodiment of the present disclosure for implementing the foregoing technical object discloses a computer program for estimating a heart age. The program may include: obtaining vital sign data of a user; and estimating a heart age of the user based on the vital sign data of the user by using a pre-trained artificial neural network model, in which the pre-trained artificial neural network model corresponds to an artificial neural network model pre-trained based on information related to a heart disease.

Another exemplary embodiment of the present disclosure for implementing the foregoing technical object discloses a computing device for estimating a heart age. The computing device includes: a processor including one or more cores; a network unit for receiving one or more vital sign data; and a memory, in which the processor obtains vital sign data of a user, and estimates a heart age of the user based on the vital sign data of the user by using a pre-trained artificial neural network model, and the pre-trained artificial neural network model corresponds to an artificial neural network model pre-trained based on information related to a heart disease.

According to the present disclosure, it is possible to estimate a heart age of the user by inputting a user's vital sign to an artificial neural network and provide analysis results, and output predictive information about a user's future heart age.

### BRIEF DESCRIPTION OF THE SEVERAL VIEWS OF THE DRAWINGS

The accompanying drawings for use in the description of the exemplary embodiments of the present disclosure are only some of the exemplary embodiments of the present disclosure, and other drawings may be obtained based on the drawings by a person of ordinary skill in the art to which the present disclosure belongs (hereinafter referred to as “a person skilled in the art”) without an effort to arrive at a novel disclosure.

FIG. 1 is a block diagram illustrating a computing device for estimating a heart age, according to an embodiment of the present disclosure.

FIG. 2 is a schematic diagram illustrating a network function according to the exemplary embodiment of the present disclosure.

FIG. 3 is a flowchart illustrating a process for transfer learning an artificial neural network model according to the embodiment of the present disclosure.

FIG. 4 is a flowchart illustrating a process for ensembling one or more artificial neural network models according to the embodiment of the present disclosure.

FIG. 5 is a schematic diagram illustrating a model that is an ensemble of one or more artificial neural network models according to the embodiment of the present disclosure.

FIG. 6 is a conceptual diagram illustrating a process for generating analytical information about a heart age of the user estimated by the artificial neural network model according to the embodiment of the present disclosure.

FIG. 7 is a conceptual diagram illustrating an example of analytical information about a heart age of the user according to the embodiment of the present disclosure.

FIG. 8 is a conceptual diagram illustrating predictive information about a user's future heart age according to the embodiment of the present disclosure.

FIG. 9 is a simple and general schematic diagram illustrating an example of a computing environment in which exemplary embodiments of the present disclosure are implementable.

### DETAILED DESCRIPTION

The present disclosure discloses a method of receiving, by an artificial neural network, vital sign as input and estimating a heart age from the input vital sign based on information related to a heart disease.

Various exemplary embodiments will now be described with reference to drawings. In the present specification, various descriptions are presented to provide appreciation of the present disclosure. However, it is apparent that the exemplary embodiments can be executed without the specific description.

“Component”, “module”, “system”, and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components.

One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein. In the present disclosure, a network function and an artificial neural network and a neural network may be interchangeably used.

In the present disclosure, real age, physical age, and chronological age may be used interchangeably.

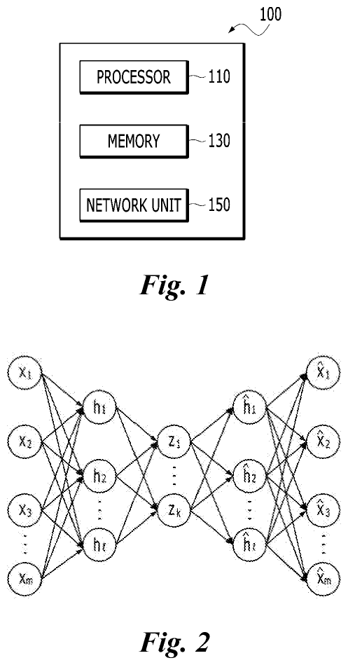

FIG. 1 is a block diagram illustrating a computing device for estimating a heart age, according to an embodiment of the present disclosure.

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100. The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for training the neural network. At least one of the CPU, GPGPU, and TPU of the processor 110 may process training of a network function. For example, both the CPU and the GPGPU may process the training of the network function and data classification using the network function. Further, in an exemplary embodiment of the present disclosure, processors of a plurality of computing devices may be used together to process the training of the network function and the data classification using the network function. Further, the computer program executed in the computing device according to an exemplary embodiment of the present disclosure may be a CPU, GPGPU, or TPU executable program.

According to the embodiment of the present disclosure, the processor 110 may estimate a heart age of the user age based on vital sign data of a user by using an artificial neural network model pre-trained based on information related to heart disease. To this end, the processor 110 may obtain vital sign data of the user. For example, the processor 110 may use the vital sign data of the user stored in the memory 130, may receive the vital sign data of the user through the network 150, and may obtain the vital sign data of the user directly from a vital sign measurement equipment (not illustrated). However, the present disclosure is not limited to the exemplified vital sign obtaining methods.

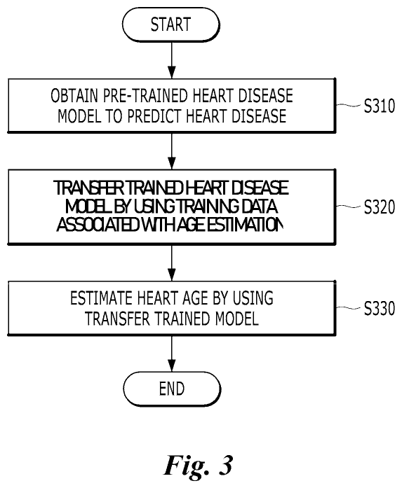

The processor 110 may pre-train an artificial neural network model for estimating a heart age to exhibit performance for the purpose by utilizing techniques, such as supervised learning, transfer learning, model ensembles, and the like. The specific process of pre-training an artificial neural network model will be described later with reference to FIGS. 3 to 5.

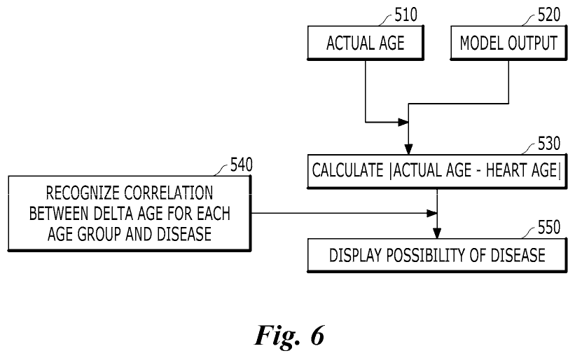

In further embodiments of the present disclosure, the processor 110 may generate analytical information about the heart age of the user age estimated by a pre-trained artificial neural network model. The analytical information about the heart age may include, but is not limited to, information about the estimated heart age of the user, information about the location of the user in the distribution of heart ages of users in the same age group, or information about comparisons to a population of users with major heart disease. The specific process of generating analytical information about a heart age of the user is described later with reference to FIG. 6.

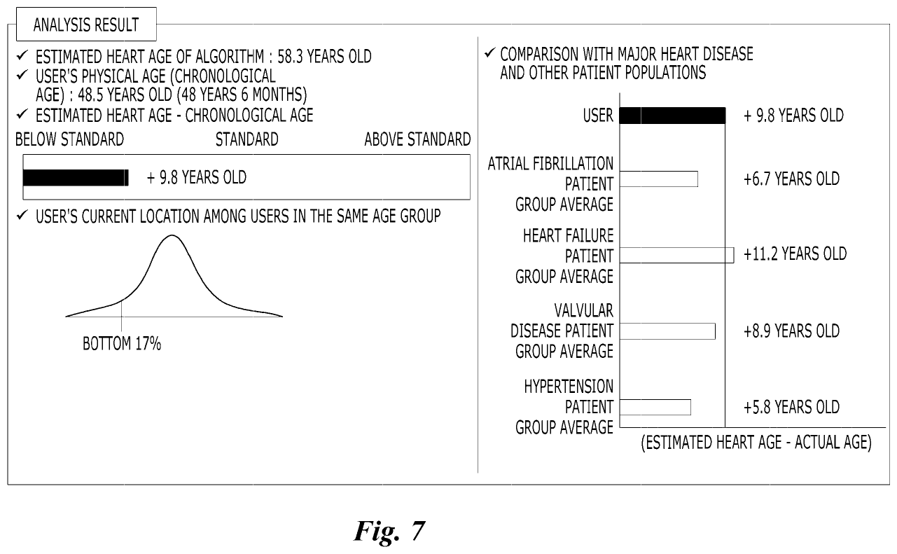

In an additional embodiment of the present disclosure, the processor 110 may generate predictive information about a user's future heart age based on the user's estimated heart age by using a pre-trained artificial neural network model. A specific method of generating predictive information about a user's future heart age and a method of training an artificial neural network model to generate the predictive information will be described below with reference to FIG. 7.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto. The network unit 150 according to several exemplary embodiments of the present disclosure may use various wired communication systems, such as a Public Switched Telephone Network (PSTN), an x Digital Subscriber Line (xDSL), a Rate Adaptive DSL (RADSL), a Multi Rate DSL (MDSL), a Very High Speed DSL (VDSL), a Universal Asymmetric DSL (UADSL), a High Bit Rate DSL (HDSL), and a local area network (LAN).

The network unit 150 presented in the present specification may use various wireless communication systems, such as Code Division Multi Access (CDMA), Time Division Multi Access (TDMA), Frequency Division Multi Access (FDMA), Orthogonal Frequency Division Multi Access (OFDMA), Single Carrier-FDMA (SC-FDMA), and other systems.

In the present disclosure, the network unit 150 may use any type of wired/wireless communication system.

The technologies described in the present specification may be used in other networks, as well as the foregoing networks.

FIG. 2 is a conceptual view illustrating a neural network according to an exemplary embodiment of the present disclosure.

A neural network model according to the exemplary embodiment of the present disclosure may include a neural network for evaluating placement of the semiconductor device. Throughout the present specification, a computation model, the neural network, a network function, and the neural network may be used as the same meaning. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship between an input node and an output node. Concepts of the input node and the output node are relative and a predetermined node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, a definition of the layer is predetermined for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

In the neural network according to an exemplary embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another exemplary embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another exemplary embodiment of the present disclosure, the number of nodes of the input layer may be larger than the number of nodes of the output layer, and the neural network may be a neural network of a type in which the number of nodes increases from the input layer to the hidden layer. The neural network according to still yet another exemplary embodiment of the present disclosure may be a neural network of a type in which the neural networks are combined.

A deep neural network (DNN) may refer to a neural network that includes a plurality of hidden layers in addition to the input and output layers. When the deep neural network is used, the latent structures of data may be determined. That is, latent structures of photos, text, video, voice, and music (e.g., what objects are in the photo, what the content and feelings of the text are, what the content and feelings of the voice are) may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, a Generative Adversarial Network (GAN), and the like. The description of the deep neural network described above is just an example and the present disclosure is not limited thereto.

In an exemplary embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be trained in at least one scheme of supervised learning, unsupervised learning, semi supervised learning, or reinforcement learning. The learning of the neural network may be a process in which the neural network applies knowledge for performing a specific operation to the neural network.

The neural network may be trained in a direction to minimize errors of an output. The training of the neural network is a process of repeatedly inputting training data into the neural network and calculating the output of the neural network for the training data and the error of a target and back-propagating the errors of the neural network from the output layer of the neural network toward the input layer in a direction to reduce the errors to update the weight of each node of the neural network. In the case of the supervised learning, the training data labeled with a ground-truth is used for each training data (i.e., the labeled training data) and in the case of the unsupervised learning, the ground-truth may not be labeled in each training data. That is, for example, the training data in the case of the supervised learning related to the data classification may be data in which category is labeled in each training data. The labeled training data is input to the neural network, and the error may be calculated by comparing the output (category) of the neural network with the label of the training data. As another example, in the case of the unsupervised learning related to the data classification, the training data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a training cycle (epoch). The learning rate may be applied differently according to the number of repetition times of the training cycle of the neural network. For example, in an initial stage of the training of the neural network, the neural network ensures a certain level of performance quickly by using a high learning rate, thereby increasing efficiency and uses a low learning rate in a latter stage of the training, thereby increasing accuracy.

In training of the neural network, the training data may be generally a subset of actual data (i.e., data to be processed using the trained neural network), and as a result, there may be a training cycle in which errors for the training data decrease, but the errors for the actual data increase. Overfitting is a phenomenon in which the errors for the actual data increase due to excessive training of the training data. For example, a phenomenon in which the neural network that trains a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the training data, regularization, dropout of omitting a part of the node of the network in the process of training, utilization of a batch normalization layer, etc., may be applied.

FIG. 3 is a flowchart illustrating a process for transfer learning an artificial neural network model according to the embodiment of the present disclosure.

In the present disclosure, supervised learning may be used as a training method for an artificial neural network model for estimating a heart age. In the case of supervised learning of an artificial neural network model, labeled data for training the artificial neural network model may be vital signs, and the vital sign may be labeled with chronological age information of the user. Through the supervised learning in this way, the artificial neural network model for estimating a heart age may learn a pattern of a user's vital sign, and in an inference operation of the artificial intelligence model, the artificial neural network model may receive the vital sign as input to infer a heart age.

In another exemplary embodiment, in the present disclosure, transfer learning may be used as a training method for the artificial neural network model for estimating a heart age together with supervised learning. Transfer learning means using some of the abilities of a neural network trained in a specific field to solve problems in a similar or different field. For example, the case where a model that has been trained to recognize dogs is additionally trained to recognize cats corresponds to the transfer learning. In general, it is known that in a situation where data is insufficient, an artificial neural network model trained through the transfer learning method performs better than an artificial neural network model trained through supervised learning alone.

In addition to the embodiment through the supervised learning of the present disclosure described above, the architecture of the artificial neural network model may be designed so that vital sign data from unhealthy users can also be used to train the artificial neural network model.

In one embodiment of the present disclosure, an artificial neural network model that has been completely pre-trained may be transfer-trained to estimate a heart age of the user. For example, as an artificial neural network model which has been completely trained, a heart disease model implemented to predict information related to a specific heart disease from vital signs may be used. However, it will be readily apparent to one of ordinary skill in the art that other models suitable for transfer learning may be used in addition to the model for predicting information related to a specific heart disease.

In operation S310, the processor 110 may obtain a heart disease model that is pre-trained to predict heart disease from the user's vital sign information. For example, the pre-trained heart disease model may be a model that receives the user's vital sign as input and outputs a probability that the user has heart failure. In this case, the type of heart disease may include, but is not limited to, arrhythmia, myocardial infarction, heart failure, and the like.

In operation S320, the processor 110 may transfer train the pre-trained heart disease model by using training data associated with the age estimation. In this case, the transfer-trained artificial neural network model may be further trained with the training data associated with the age estimation to tune the weight of the pre-trained model, and as a result, the transfer-trained artificial neural network model may recognize a correlation between a heart age and heart disease.

In operation S330, the processor 110 may estimate the heart age by using the transfer trained model.

In this way, the transfer-trained artificial neural network model 300 may use not only vital sign data from healthy users, but also vital sign data from users with specific heart disease as training data. In conclusion, the transfer-trained artificial neural network model as disclosed herein may become a robust model for accurately inferring heart age in response to the input of more diverse types of vital sign information in the inference operation.

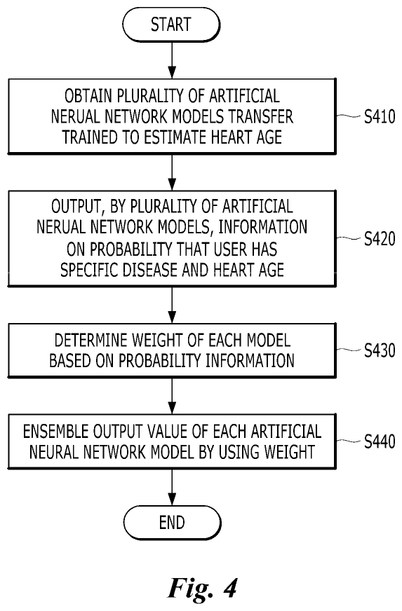

FIG. 4 is a flowchart illustrating a process for ensembling one or more artificial neural network models according to the embodiment of the present disclosure.

Ensemble learning refers to machine learning techniques that combine multiple decision trees to exhibit better performance than a single decision tree. Bagging, boosting, and voting are well-known examples of the ensemble learning.

The artificial neural network model transfer-trained to estimate a heart age by using the artificial neural network model that has been pre-trained to predict a particular heart disease is more robust than a simply supervised artificial neural network model with respect to the input of vital signs from users who are likely to have the corresponding particular disease, but the accuracy of the heart age information estimated by the corresponding artificial neural network model may be less with respect to vital sign inputs from users who may have other diseases.

In operation S410, the processor 110 may obtain a plurality of artificial neural network models that are transfer-trained to estimate a heart age. For example, a model transfer-trained to estimate a heart age from a model pre-trained to output a probability that a user has arrhythmia may be obtained.

In operation S420, the plurality of artificial neural network models may each output, by the processor 110, probability information that the user has a particular disease and heart age. For example, a model transfer-trained from a model that is pre-trained to output a probability that the user has arrhythmia is one of the plurality of artificial neural network models and may output information about the probability that the user has an arrhythmia and the heart age of the user from the user's vital signs.

In operation S430, the processor 110 may determine a weight for each model based on the probability information output by the plurality of artificial neural network models in operation S420. For example, if there is a 40% probability that the user has arrhythmia and the heart age of the user age output by the model to which the model pre-trained to output the probability that the user has arrhythmia is transfer trained is 42 years old, a weight of 40% may be given to the model's output value of 42 years old.

In operation S440, the processor 110 may ensemble the output values of the artificial neural network models by using the determined weights, respectively. For example, it may be assumed that there is a 40% probability that the user has arrhythmia and the heart age of the user output by the model transfer trained from the model pre-trained to output the probability that the user has arrhythmia is 42 years old, and there is a 10% probability that the user has an myocardial infarction and the heart age of the user output by the model transfer trained from the model pre-trained to output the probability that the user has an myocardial infarction is 50 years old. In this case, the output value of the ensembled model may be 43.6 years old, which is a weighted average of 42 years old and 50 years old by reflecting the respective weights. However, the method of ensembling the output value of the model in the present disclosure is not limited to weighted averaging as in this example.

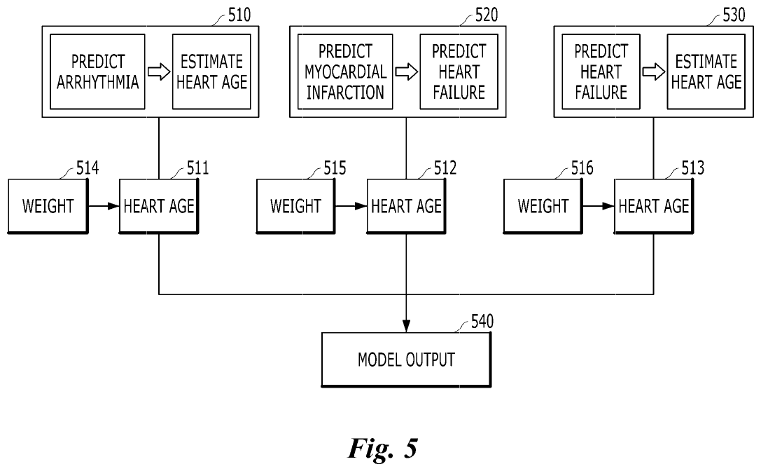

FIG. 5 is a schematic diagram illustrating a model that is an ensemble of one or more artificial neural network models according to the embodiment of the present disclosure.

In an additional embodiment of the present disclosure, the artificial neural network model for estimating a heart age may be an ensembled model of a plurality of artificial neural network models that are transfer-trained based on the artificial neural network model that is pre-trained to predict a particular heart disease. For example, the artificial neural network model for estimating a heart age may be an ensembled model of a model 510 to which a model pre-trained to predict arrhythmia is transfer trained, a model 520 to which a model pre-trained to predict myocardial infarction is transfer trained, and a model 530 to which a model pre-trained to predict heart failure is transfer trained.

Specifically, an output 540 of the artificial neural network model for estimating the heart age may be a combined value of the output values of the plurality of artificial neural network models 510, 520, and 530 trained through transfer learning based on a plurality of heart disease models implemented to predict a particular disease.

For example, each of the plurality of transfer-trained artificial neural network models 510, 520, and 530 may derive probability information indicating that the user has a particular disease and heart age information from the input vital sign. Thereafter, the plurality of transfer-trained artificial neural network models 510, 520, and 530 may specify weights 514, 515, and 516 for the respective models based on the probability information. Then, by using the specified weights based on the probability information, the heart ages 511, 512, and 513 estimated by the plurality of transfer-trained artificial neural network models 510, 520, and 530, respectively, may be weighted averaged to become the output value 540 of the final model. The structure of ensembling a plurality of transfer-trained artificial neural network models in the present disclosure is not limited to the method of the above example, and other techniques, such as bagging, boating, and boosting, may be used.

According to the above example, when a user has a high probability of a particular disease, for example, when a user has a high probability of arrhythmia, the heart age 511 estimated by the model 510 transfer trained from the arrhythmia prediction model may have a high weight in the overall model outputs because the weight 514 of the model 510 transfer trained from the arrhythmia prediction model is calculated to be large in the outputs of the total models.

By ensembling the plurality of transfer-trained models as described above, the finalized heart age estimation model may be more robust to vital sign inputs of users with different types of diseases, resulting in higher model performance.

FIG. 6 is a conceptual diagram illustrating a process for generating analytical information about a heart age of the user age estimated by the artificial neural network model according to the embodiment of the present disclosure.

The processor 110 may provide analytical information about the user's degree of risk for heart disease and need for management in relation to the heart age of the user.

Specifically, in the training operation of the artificial neural network model, the processor 110 may train the artificial neural network with data in which the vital sign is labeled with diseases and chronological ages. For example, the processor 110 may train the artificial neural network model to estimate a heart age, calculate the difference between the estimated heart age and the user's chronological age, and recognize a correlation between the difference and each disease (640).

In the inference operation of the artificial neural network model, the processor 110 may receive vital data as input and estimate a heart age as an output 620 of the artificial neural network model by using the artificial neural network model. The processor 110 may compare the estimated heart age to the actual age 610, that is, the chronological age, to calculate a difference (delta age) 630. Since the artificial neural network has been trained 640 to recognize the correlation between the difference and each disease, the artificial neural network may analyze the probability 650 of a disease for the input vital sign data. The analysis results of the above artificial neural network model may be visualized and displayed to the user in the form of FIG. 6.

FIG. 7 is a conceptual diagram illustrating an example of analytical information about a heart age of the user age according to the embodiment of the present disclosure.

As an example of analytical information, the processor 110 may present the heart age of the user age estimated by the artificial neural network model, the user's chronological age, and the difference between the heart age of the user and the chronological age.

The processor 110 may also calculate and display to the user where the heart age of the user lies within a normalized distribution created by collecting the heart age estimation results of users in the same age group. For example, the processor 110 may display analytical information to the user in the form of “Your estimated heart age is 9.8 years older than your chronological age, which places you in the bottom 17% of users in the same age group”.

The processor 110 may display the level of the heart age of the user by comparing the heart age of the user with the heart age of a group of users with major heart diseases. For example, the processor 110 may generate a graph, such as the one illustrated in FIG. 6, indicating that the user's estimated heart age is 9.8 years older than the user's chronological age, and may provide an accompanying analytical result showing figures for patients with heart disease, such as patients with atrial fibrillation, for reference. As described above, information using the difference between the user's estimated heart age and the users chronological age is displayed and is compared with others' information, so that it is possible to help the user or medical teams to determine the possibility of the heart disease of the user and the action required or not.

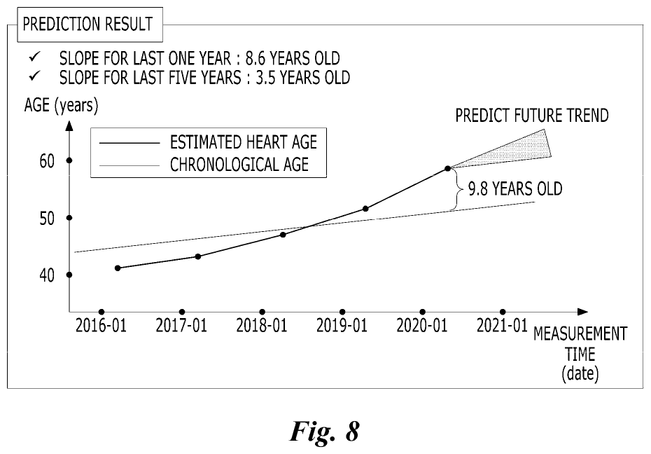

FIG. 8 is a conceptual diagram illustrating predictive information about a user's future heart age according to the embodiment of the present disclosure.

The processor 110 may generate predictive information about a user's future heart age based on a user's estimated heart age by using an artificial neural network model. In this case, as a detailed operation of generating the predictive information, whether data according to the measurement of the user's vital sign in the past exists may be first checked. When the user's past vital sign data exists, the processor 110 may estimate the heart age at each measurement time point in the past from the past vital sign data and estimate a current heart age by using the artificial neural network model. The artificial neural network may then generate predictive information of a future heart age from the past heart age and the current heart age. The predictive information may be expressed as a specific point at a specific time point, and may be expressed as an area plotting possible slope values, but the present disclosure is not limited thereto.

The processor 110 may calculate and display to the user a number, such as the degree of change in the estimated heart age in recent years from the estimated values of the heart age in the past, and the difference between the chronological age and the heart age at each time point. For example, as illustrated in FIG. 7, the processor 110 may provide the user with information that the heart age has been increasing at a rate of 8.6 years per year in the most recent year, and at a rate of 3.5 years per year in the most recent five years.

The past vital sign data for the same user may be collected at regular intervals or may be collected at irregular intervals, that is, different time intervals between data. When time intervals between the points configuring time-series data are different, an artificial neural network model to predict future information may be beneficial.

In the present disclosure, an artificial neural network model that generates predictive information about a future heart age by the processor 110 may be trained through supervised learning. The training data for supervised learning may include a plurality of vital sign data measured at a particular time period, and the ground-truth labels for the training data may include heart age information associated with a time point after a predetermined time from a corresponding specific time. By building the pair of the input data and the ground-truth label as training data, and then training an artificial neural network model that includes a long short-term memory (LTSM) or gated recurrent unit (GRU), the artificial neural network model may calculate a future estimated value from the past data.

In models dealing with time series data, it is well known that if there are missing data points, or empty data points due to irregular measurement time intervals in the data, the training of a model with such data will result in poor performance. In the present disclosure and other inventions in the field of vital signs, user's vital sign are often measured at irregular time intervals, so solving the problem of missing or empty data may be an important challenge for improving model performance. To solve the problem, a method of preprocessing data to improve a learning effect, a method of constructing and training a model of an artificial neural network to prevent performance degradation without any preprocessing, or a combination thereof may be used.

For example, the artificial neural network model that generates predictive information about the future heart age of the present disclosure may be designed to include an interpolation network. In this case, the artificial neural network model interpolates (imputates) the data of the empty point and then learns from the interpolated data and the data that already exists.

As another example, a decay rate mechanism may be introduced into the input layer and the hidden layer of an artificial neural network model that is trained with time series data measured at irregular intervals. The decay rate may be determined by prior knowledge, modeling, or data-based learning as a learnable parameter of how other actual measured data affect missing or empty data based on time-interval information. Those skilled in the art will be able to construct an artificial neural network model having the corresponding features without much difficulty from the description of the interpolation network and decay rate mechanisms described in the present disclosure.

The preprocessing and the model structure of the present disclosure allow the model to be trained appropriately despite the fact that the user's vital signs were measured data at irregular time intervals.

Disclosed is a computer readable medium storing the data structure according to an exemplary embodiment of the present disclosure.

The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device can perform operations while using the resources of the computing device to a minimum. Specifically, the computing device can increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO-Last in First Out) in which the data is input last and output first. The queue is a data listing structure that may access data limitedly and unlike a stack, the queue may be a data structure (FIFO-First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an active function associated with each node or layer of the neural network, and a loss function for training the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an active function associated with each node or layer of the neural network, and a loss function for training the neural network or a combination thereof. In addition to the above-described configurations, the data structure including the neural network may include predetermined other information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called nodes. The nodes may also be called neurons. The neural network is configured to include one or more nodes.

The data structure may include data input into the neural network. The data structure including the data input into the neural network may be stored in the computer readable medium. The data input to the neural network may include training data input in a neural network training process and/or input data input to a neural network in which training is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example and the present disclosure is not limited thereto. The data structure may include the weight of the neural network (in the present disclosure, the weight and the parameter may be used as the same meaning). In addition, the data structures, including the weight of the neural network, may be stored in the computer readable medium. The neural network may include a plurality of weights. The weight may be variable and the weight is variable by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example and the present disclosure is not limited thereto.

As a non-limiting example, the weight may include a weight which varies in the neural network training process and/or a weight in which neural network training is completed. The weight which varies in the neural network training process may include a weight at a time when a training cycle starts and/or a weight that varies during the training cycle. The weight in which the neural network training is completed may include a weight in which the training cycle is completed. Accordingly, the data structure including the weight of the neural network may include a data structure including the weight which varies in the neural network training process and/or the weight in which neural network training is completed. Accordingly, the above-described weight and/or a combination of each weight are included in a data structure including a weight of a neural network. The data structure is just an example and the present disclosure is not limited thereto.

The data structure including the weight of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weight of the serialized neural network may be reconfigured in the same computing device or another computing device through deserialization. The data structure including the weight of the neural network is not limited to the serialization. Furthermore, the data structure including the weight of the neural network may include a data structure (for example, B-Tree, Trie, m-way search tree, AVL tree, and Red-Black Tree in a nonlinear data structure) to increase the efficiency of operation while using resources of the computing device to a minimum. The above-described matter is just an example and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of training cycle iterations, weight initialization (for example, setting a range of weight values to be subjected to weight initialization), and Hidden Unit number (e.g., the number of hidden layers and the number of nodes in the hidden layer). The data structure is just an example and the present disclosure is not limited thereto.

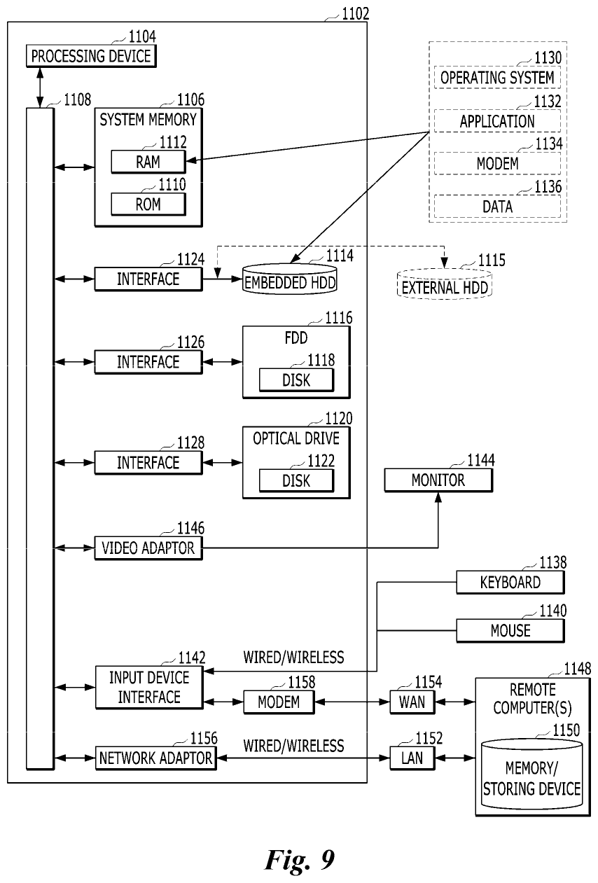

FIG. 9 is a normal and schematic view of an exemplary computing environment in which the exemplary embodiments of the present disclosure may be implemented.

It is described above that the present disclosure may be generally implemented by the computing device, but those skilled in the art will well know that the present disclosure may be implemented in association with a computer executable command which may be executed on one or more computers and/or in combination with other program modules and/or a combination of hardware and software.

In general, the program module includes a routine, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devices as well as a single-processor or multi-processor computer system, a mini computer, and a main frame computer.

The exemplary embodiments described in the present disclosure may also be implemented in a distributed computing environment in which predetermined tasks are performed by remote processing devices connected through a communication network. In the distributed computing environment, the program module may be positioned in both local and remote memory storage devices.

The computer generally includes various computer readable media. Media accessible by the computer may be computer readable media regardless of types thereof and the computer readable media include volatile and non-volatile media, transitory and non-transitory media, and mobile and non-mobile media. As a non-limiting example, the computer readable media may include both computer readable storage media and computer readable transmission media. The computer readable storage media include volatile and non-volatile media, transitory and non-transitory media, and mobile and non-mobile media implemented by a predetermined method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined other media which may be accessed by the computer or may be used to store desired information, but are not limited thereto.

The computer readable transmission media generally implement the computer readable command, the data structure, the program module, or other data in a carrier wave or a modulated data signal such as other transport mechanism and include all information transfer media. The term “modulated data signal” means a signal acquired by setting or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An exemplary environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an interior hard disk drive (HDD) 1114 (for example, EIDE and SATA), in which the interior hard disk drive 1114 may also be configured for an exterior purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD, and the like). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. An interface 1124 for implementing an exterior drive includes at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined data in an appropriate digital format. In the description of the computer readable media, the mobile optical media such as the HDD, the mobile magnetic disk, and the CD or the DVD are mentioned, but it will be well appreciated by those skilled in the art that other types of media readable by the computer such as a zip drive, a magnetic cassette, a flash memory card, a cartridge, and others may also be used in an exemplary operating environment and further, the predetermined media may include computer executable commands for executing the methods of the present disclosure. Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a computing device computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158 or has other means that configure communication through the WAN 1154 such as connection to a communication computing device on the WAN 1154 or connection through the Internet. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is exemplary and other means configuring a communication link among computers may be used.

The computer 1102 performs an operation of communicating with predetermined wireless devices or entities which are disposed and operated by the wireless communication, for example, the printer, a scanner, a desktop and/or a portable computer, a portable data assistant (PDA), a communication satellite, predetermined equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11(a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It will be appreciated by those skilled in the art that information and signals may be expressed by using various different predetermined technologies and techniques. For example, data, instructions, commands, information, signals, bits, symbols, and chips which may be referred in the above description may be expressed by voltages, currents, electromagnetic waves, magnetic fields or particles, optical fields or particles, or predetermined combinations thereof.

It may be appreciated by those skilled in the art that various exemplary logical blocks, modules, processors, means, circuits, and algorithm steps described in association with the exemplary embodiments disclosed herein may be implemented by electronic hardware, various types of programs or design codes (for easy description, herein, designated as software), or a combination of all of them. In order to clearly describe the intercompatibility of the hardware and the software, various exemplary components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various exemplary embodiments presented herein may be implemented as manufactured articles using a method, a device, or a standard programming and/or engineering technique. The term manufactured article includes a computer program, a carrier, or a medium which is accessible by a predetermined computer-readable storage device. For example, a computer-readable storage medium includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. Further, various storage media presented herein include one or more devices and/or other machine-readable media for storing information.

It will be appreciated that a specific order or a hierarchical structure of steps in the presented processes is one example of exemplary accesses. It will be appreciated that the specific order or the hierarchical structure of the steps in the processes within the scope of the present disclosure may be rearranged based on design priorities. Appended method claims provide elements of various steps in a sample order, but the method claims are not limited to the presented specific order or hierarchical structure.

The description of the presented exemplary embodiments is provided so that those skilled in the art of the present disclosure use or implement the present disclosure. Various modifications of the exemplary embodiments will be apparent to those skilled in the art and general principles defined herein can be applied to other exemplary embodiments without departing from the scope of the present disclosure. Therefore, the present disclosure is not limited to the exemplary embodiments presented herein, but should be interpreted within the widest range which is coherent with the principles and new features presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method performed by a computing device for estimating a heart age, the method comprising:
obtaining vital sign data of a user; and
estimating a heart age of the user based on the vital sign data of the user by using a pre-trained artificial neural network model,
wherein the pre-trained artificial neural network model corresponds to an artificial neural network model pre-trained based on information related to a heart disease.

2. The method of claim 1, wherein the pre-trained artificial neural network model corresponds to an artificial neural network model pre-trained by supervised learning, and
wherein training data for the supervised learning includes:
input data including measured vital sign data; and
a ground-truth label including age information of the user associated with the measured vital sign data.

3. The method of claim 1, wherein the pre-trained artificial neural network model corresponds to an artificial neural network model trained through transfer learning based on a heart disease model implemented to predict heart disease.

4. The method of claim 3, wherein the pre-trained artificial neural network model corresponds to an artificial neural network model transfer-trained based on operations of:
obtaining a trained heart disease model; and
tuning a weight of the trained heart disease model by using training data associated with age estimation.

5. The method of claim 3, wherein the pre-trained artificial neural network model includes a plurality of artificial neural network models trained through transfer learning based on a plurality of heart disease models implemented to predict a plurality of heart diseases, respectively, and
an architecture of the pre-trained artificial neural network model that includes an architecture of ensembling of the plurality of artificial neural network models.

6. The method of claim 5, wherein the architecture of ensembling of the plurality of artificial neural network models includes:
an artificial neural network architecture for designating a plurality of probabilities for the plurality of heart diseases as weights, and
weighted averaging output values of the plurality of artificial neural network models by using the weights.

7. The method of claim 1, further comprising:
generating analytical information about the heart age of the user estimated by the pre-trained artificial neural network model,
wherein the analytical information includes:
information about the heart age of the user estimated by the pre-trained artificial neural network model;
information about a position of the user in a distribution of heart ages of users in a same age group; and
information about a comparison with a group of users with major heart diseases.

8. The method of claim 1, further comprising:
generating predictive information about a future heart age of the user based on the estimated heart age of the user by using the pre-trained artificial neural network model.

9. The method of claim 8, wherein the generating of the predictive information about the future heart age of the user based on the estimated heart age of the user by using the pre-trained artificial neural network model includes:
generating predictive information about a future heart age based on the estimated heart age information and past vital sign data by using the pre-trained artificial neural network model.

10. The method of claim 9, wherein the past vital sign data is measured at different time intervals.

11. The method of claim 9, wherein the pre-trained artificial neural network model corresponds to an artificial neural network model pre-trained by supervised learning, and
wherein the training data for the supervised learning includes:
input data including a plurality of vital sign data measured over a specific time period; and
a ground-truth label that includes heart age information associated with a time point after a predetermined time from the specific time period, to output predictive information about a future heart age.

12. The method of claim 9, wherein the pre-trained artificial neural network model corresponds to an artificial neural network model pre-trained with least one of operations of:
imputating the past vital sign data of the user by using an interpolation network and applying the imputed past vital sign data to training; or
introducing a decay rate into an input layer and a hidden layer of the artificial neural network model.

13. A computer program stored in a non-transitory computer-readable storage medium including instructions for causing a computing device to perform operations, the operations comprising:
obtaining vital sign data of a user; and
estimating a heart age of the user based on the vital sign data of the user by using a pre-trained artificial neural network model,
wherein the pre-trained artificial neural network model corresponds to an artificial neural network model pre-trained based on information related to a heart disease.

14. A computing device, comprising:
a processor including one or more cores;
a network unit for receiving one or more vital sign data; and
a memory coupled to the processor,
wherein the processor is configured to:
obtain vital sign data of a user, and
estimate a heart age of the user based on the vital sign data of the user by using a pre-trained artificial neural network model, and
wherein the pre-trained artificial neural network model corresponds to an artificial neural network model pre-trained based on information related to a heart disease.
